(19) United States
(12) Patent Application Publication
(10) Pub. No.: US 2009/0069766 A1
(43) Pub. Date: Mar. 12, 2009

(54) PERSONAL ABSORBENT PAD

(76) Inventor: Barbara J. Bannister, Mill Creek, WA (US)

Correspondence Address: Barbara J. Bannister, 117 Poppy Road, Mill Creek, WA, 98012, US

(21) Appl. No.: 12/118,906

(22) Filed: May 12, 2008

Related U.S. Application Data
(60) Provisional application No. 60/917,712, filed on May 14, 2007.

(51) Int. Cl. A61F 13/15; A41C 3/00; A41D 27/26

(52) U.S. Cl. 604/359; 604/358; 450/1; 2/267; 604/385.03

(57) ABSTRACT

A personal absorbent pad that includes a front panel, a back panel, two sides, and an adornment. The two sides are configured to fit between a pair of breasts so as to hold the pad in place. The absorbent pad has an adornment.

### REFERENCE TO RELATED APPLICATION

This application claims priority to U.S. provisional patent application Ser. No. 60/917,712, filed May 14, 2007, and incorporated herein by reference.

### BACKGROUND OF THE INVENTION

Chest perspiration is often a problem, especially in hot or humid conditions. In particular, perspiration may form between a person's breasts, causing an uncomfortable condition. In addition, the presence of a brassiere and/or shirt may create difficulty in managing chest perspiration.

### SUMMARY OF THE INVENTION

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description of some embodiments that are presented later.

The present invention provides a personal absorbent pad having a front panel, a back panel, a top edge, and left and right side edges. In an embodiment, the back panel, the left edge, and the right edge are contoured so that, when the absorbent pad is worn by a user, the back panel fits against and extends along a portion of the chest of the user, the right edge fits against and extends along a portion of the right breast of the user, and the left edge fits against and extends along a portion of the left breast of the user. When worn, the absorbent pad absorbs perspiration as it flows down the chest, preventing or limiting the flow of perspiration through the breasts.

In an embodiment, the front panel and back panel may be opposite sides of a single flexible, absorbent material, or may be two different materials attached together. As an example, the two materials may be stacked against each other and sewn along their edges. The left and right side edges are defined along the left and right edges of the front and side panels.

In an embodiment, the top edge of the absorbent pad includes an adornment, such as lace. The top edge may be straight or contoured to a decorative shape. Other portions of the absorbent pad may be decorated or adorned, including but not limited to the front panel.

The absorbent pad may be attached to clothing or directly to the wearer. In an embodiment, the absorbent pad fits between a brassiere and a wearer's chest.

Other features of the invention will become apparent from the following detailed description when taken in conjunction with the drawings, in which:

### BRIEF DESCRIPTION OF THE DRAWINGS

FIG. 1 shows a front perspective view of an absorbent pad, as worn by a person, in accordance with an embodiment.

FIG. 2 shows a front view of the absorbent pad of FIG. 1.

FIG. 3 shows a side view of the absorbent pad of FIG. 1.

FIG. 4 shows a side view of an absorbent pad in accordance with an alternate embodiment.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

Referring now to the drawings, in which like reference numerals represent like parts throughout the several views, FIG. 2 shows an absorbent pad 22 in accordance with an embodiment of the invention. The absorbent pad 22 is designed to be worn by a woman, but may be worn by anyone.

In accordance with an embodiment of the invention, an absorbent pad 22 includes a front panel 30 and a back panel 32. The front panel 30 and back panel 32 may be made of any flexible material. In an embodiment, the back panel 32 is absorbent. The front panel 30 may also be absorbent, but is not necessarily so. Suitable materials for the back panel 32 (and the front panel, if absorbent) include cotton, silk, satin, polyester, nylon, linen, terrycloth, or synthetic materials having the ability to wick away moisture from the body. The front panel 30 and back panel 32 may be formed from the same material, or may be formed from different materials. The front panel 30 and the back panel 32 may also be formed from a single piece of material, wherein one side defines the front and the opposite side defines the back.

In the embodiment shown in the drawings, the absorbent pad 22 forms a four-sided figure, with top and bottom edges 36, 38 being straight, although other curves may be used for these edges. The top edge 36, for example, may be convex in shape so as to form a convex rounded appearance and to extend further up the chest of a wearer. The top edge 36 may also form a concave rounded appearance in order expose more cleavage.

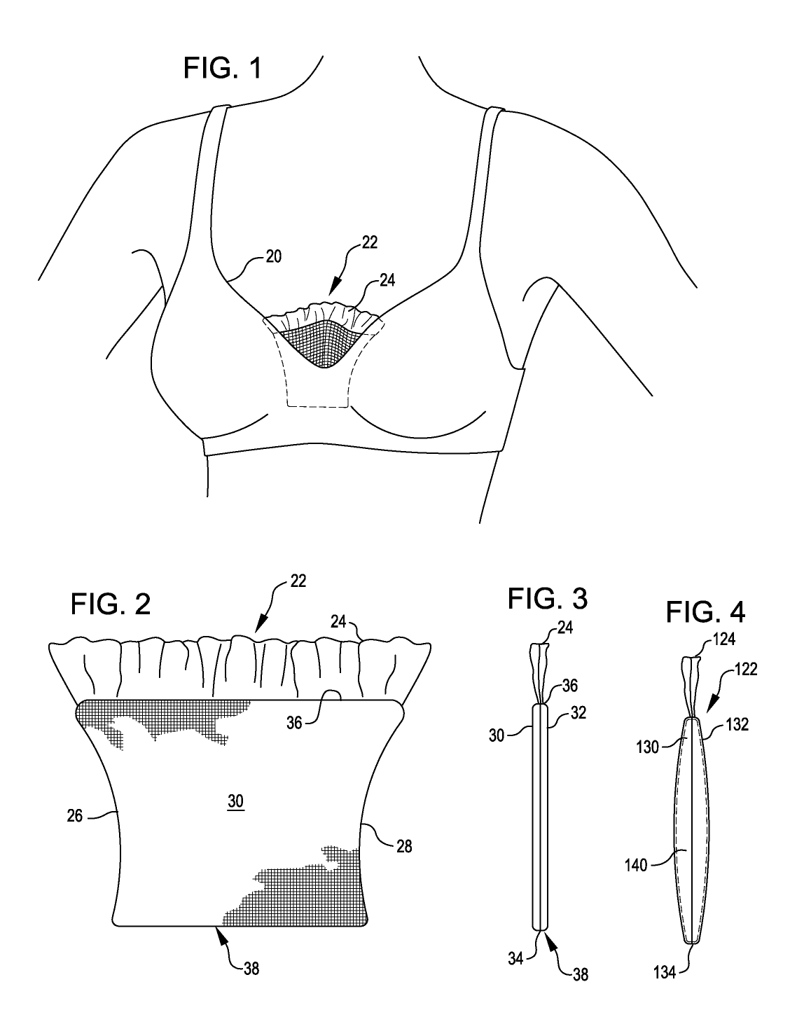

In the embodiment shown in FIG. 2, the front panel 30 and the back panel 32 have concave left and right side edges 28, 26, although other shapes are possible. In an embodiment, as shown in FIG. 1, the curve of the concave side edges 28, 26 is such that, when the back panel 32 is aligned against the chest of a wearer, the absorbent pad 22 fits in the space between and against and along the two breasts of a wearer. More specifically, when the absorbent pad 22 is in place between the breasts of a wearer, the left side edge 28 fits against and extends along a portion of the left breast of the wearer, and the right side edge 26 fits against and extends along a portion of the right breast of the user.

As is known, a V-shaped depression, called “cleavage” is formed at the intersection between a woman's breasts. The chest forms the back portion of this V. The absorbent pad 22 is in an embodiment formed so that it engages all sides of the V, including both breasts and the chest, when worn. The absorbent pad 22 aligns in a parallel fashion against the chest. The concave left and right side edges 28, 26 fit against the breasts.

As shown in FIG. 1, the absorbent pad 22 may be held in place by a brassiere 20, although the absorbent pad may be held in place in another manner. For example, the absorbent pad may include hooks (not shown) for attaching to a brassiere. Other ways of attaching the absorbent pad 22 to a brassiere, or other clothing include snaps, hook and loop fasteners, pins, tape, glue, clips, clasps, clamps, magnets, and, in general, any suitable fastener for attaching the absorbent pad 22 to another object. Hooks or other devices (not shown) may also simply restrict movement of the absorbent pad 22, for example, by hooking over the top of a brassiere. The absorbent pad 22 may also include a sticky substance such as tape or glue for securing the absorbent pad to the chest of the user so that the absorbent pad 22 need not be secured in place by a brassiere.

Supporting the absorbent pad 22 in such a position has many advantages. For example, an absorbent pad 22 helps control perspiration. In particular, an absorbent pad 22 in a position between two breasts absorbs perspiration and is also in a position so as to be easily handled by a user who may manipulate the absorbent pad 22 to wipe away perspiration between the breasts in a discrete manner. The absorbent pad 22 may also be removed from between the breasts in order to be used to wipe away perspiration on other parts of a user's body. In addition, an absorbent pad 22 can be used to hide cleavage.

A manufacturer wishing to produce absorbent pads may produce many absorbent pads with curves configured to fit a common breast size, or the manufacture may sell absorbent pads with varying curves to fit a variety of breast sizes. The size of the front panel 30 and the back panel 32 may also vary. For example, the distance between the curves of the panels may vary to accommodate a variety of people having different distances between their breasts. In addition, the length of the front panel 30 and back panel 32 between the flat sides may vary as well.

Also shown in FIG. 2, the absorbent pad 22 may have an adornment 24. In an embodiment the adornment 24 comprises a lace ribbon and extends along the top edge 32 of the absorbent pad 22, although an adornment or other decoration may be utilized on other locations on the absorbent pad, including the front panel. Adornments may be made of other materials such as ribbon, fur, faux fur, or anything suitable for adding decoration or ornamentation. Adornments may also include other features such as lights, beads, sequins, or anything decorative or ornamental.

In an embodiment, the front panel 30 is constructed from an aesthetically pleasing material, for example, of material of a color meant to match a particular outfit. In addition, other materials may be used, such as material having camouflage for soldiers, bright colors for construction workers, reflective material for people exercising outside, and colors and/or logos of favorite sports teams. In general, the materials used may be functional, aesthetic, or both. In an alternate embodiment, the front panel 30 and the back panel 32 have a different appearance so that a user may reverse the absorbent pad 22 so as to choose which panel to display. For example, the front panel 30 may be yellow while the back panel 32 is green so that a user may choose to display either yellow or green. A front panel 30 or a back panel 32 may also have more than one color, or a design.

The absorbent pad 22 may be constructed by aligning the front panel 30 and back panel 32 and sewing them together almost completely around the perimeter, leaving a portion of the perimeter unsewn. Then the absorbent pad 22 may be turned inside out through the unsewn portion, thus moving the sewn edges to the inside of the absorbent pad and forming a seam 34. The unsewn portion then may be stitched together. If an adornment 24 and/or additional material (such as the stuffing 140, described below) is desired for the absorbent pad 22, those materials may be placed in a position such that they are properly oriented and in the proper position when the panels are turned inside out through the unsewn portion.

Other methods of constructing an absorbent pad may also be used. For example, instead of sewing a front panel 30 to a back panel 32, the absorbent pad may be made from a single piece of material. For instance, a piece of thick material having the shape of the absorbent pad shown in FIG. 2 may be provided. In addition, a single piece of material may be provided having a geometry similar to a skull cap such that only one side needs to be sewn in the construction of the absorbent pad. Of course, more pieces of material than shown in the drawings and described above may also be used.

Another possible feature that may be incorporated into an absorbent pad 22 is the addition of pockets. For example, an absorbent pad 22 may include a pocket for holding a fragrant substance. An absorbent pad may also include a pocket for holding items such as keys or money. Pockets may include features such as buttons, zippers, hook and loop fasteners, or other fasteners for keeping a pocket closed.

FIG. 4 shows an alternate embodiment of an absorbent pad 122 in accordance with an embodiment. Like the absorbent pad 22, the absorbent pad 122 may have a front panel 130, a back panel 132, and an adornment 124 that are similar to the front panel 30, back panel 32, and adornment 24, respectively. As shown in the drawing, the absorbent pad 122 may have additional material, stuffing 140, between the front panel 130 and the back panel 132. In an embodiment, the stuffing 140 is absorbent such as cotton batting or batting made from other materials. Stuffing 140 may also emit a fragrance, for example, by including fragrant oils. Suitable materials for the stuffing 140 include all the materials disclosed above. Having additional material gives the absorbent pad 22 the ability to absorb excess perspiration and also gives the absorbent pad 22 a greater thickness, which may be aesthetically desirable. Stuffing may be, but need not be, removable.

Any absorbent pad, such as the absorbent pad 22 or the absorbent pad 122 may include other features. For example, an absorbent pad may include wires, plastic inserts, or other mechanisms for maintaining the absorbent pad in a particular shape. For instance, wires may be incorporated into an absorbent pad in order to give the front or back surface a curve, which may closely match a curve of a user's body between his or her breasts.

Other variations are within the spirit of the present invention. Thus, while the invention is susceptible to various modifications and alternative constructions, a certain illustrated embodiment thereof is shown in the drawings and has been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A personal absorbent pad, comprising:
a front panel;
a back panel;
a top edge;
left and right concave side edges;
the back panel, the left edge, and the right edge being contoured so that, when the absorbent pad is worn by a user, the back panel fits against and extends along a portion of the chest of the user, the right edge fits against and extends along a portion of the right breast of the user, and the left edge fits against and extends along a portion of the left breast of the user.

2. The pad of claim 1, wherein the front panel and back panel are opposite sides of a single flexible, absorbent material.

3. The pad of claim 1, wherein the front panel and back panel are two different materials attached together.

4. The pad of claim 1, further comprising an adornment extending along the top edge.

5. The pad of claim 1, further comprising a connector, the connector configured to secure the pad to a brassiere or other piece of clothing.

6. The pad of claim 1, further comprising stuffing between the front and back panels.

7. The pad of claim 6, wherein the stuffing emits a fragrance.

8. The pad of claim 1, wherein the absorbent pad is configured such that, when in place between the breasts of a wearer, the left side edge fits against and extends along a portion of a left breast of the wearer, and the right side edge fits against and extends along a portion of a right breast of the wearer.

9. The pad of claim 1, wherein the front panel and the back panel comprise different decorative appearances.

10. The pad of claim 1, wherein the front panel is decorated with camouflage.

11. The pad of claim 1, wherein the front panel is decorated with reflective material.

12. The pad of claim 1, wherein the front panel is decorated with a logo.

13. The pad of claim 1, further comprising a pocket on the pad.

14. The pad of claim 1, further comprising an adornment attached to the pad.

15. The pad of claim 14, wherein the adornment comprises a light.

16. The pad of claim 14, wherein the adornment comprises beads.

17. The pad of claim 14, wherein the adornment comprises sequins.
